(19) United States
(12) Patent Application Publication
(10) Pub. No.: US 2011/0028758 A1
(43) Pub. Date: Feb. 3, 2011

(54) Trans-cinnamic acid derivative, its preparation mathod and the use

(76) Inventors: Wei Wang, Guangdong (CN); Jun Ying, Guangdong (CN); Wenzhan Chen, Guangdong (CN); Xuebin Liu, Guangdong (CN); Kui Xu, Guangdong (CN); Qingchun Ni, Guangdong (CN)

Correspondence Address: DAVID AND RAYMOND PATENT FIRM, 108 N. YNEZ AVE., SUITE 128, MONTEREY PARK, CA, 91754, US

(21) Appl. No.: 12/311,204

(22) PCT Filed: Jun. 4, 2008
(86) PCT No.: PCT/CN2008/001089

§ 371 Date: Nov. 12, 2009

(30) Foreign Application Priority Data
Jun. 15, 2007 (CN) 200710028602.8

(51) Int. Cl. C07C 59/64; C07C 51/09

(52) U.S. Cl. 562/478

(57) ABSTRACT

A compound of formula (I) or its pharmaceutically acceptable salt is prepared by ring opening of osthol under basic condition. The compound of formula (I) or its pharmaceutically acceptable salt has activity of selectively inhibiting tumor cells and lower toxicity, and can be used for preparing anti-tumor drugs.

### BACKGROUND OF THE PRESENT INVENTION

1. Field of Invention

The present invention relates to a trans-cinnamic acid derivative and the preparation method and the application thereof.

2. Description of Related Arts

Tumor is a serious disease which could damage the health of human body. Tumor prevention and control work has been the focus of the field of medical research. At present, because the industrial development brought many environmental pollution problems, the survival of mankind environmental quality is declining so as to cause the rising rate of the tumor-related disease morbidity and mortality. Radiotherapy and chemotherapy are currently the main means for the treatment of tumor. The normal cells in the human body are being inhibited while the radiotherapy and chemotherapy is inhibiting the cancer cells, so that the immunity of the human body is reduced to cause new complications. The medicine for the treatment of tumor-related diseases is not satisfied. The selectivity of current clinical use of cytotoxic drugs is not high to recognize the target tumor cells, and leading to the vicious destruction of normal cells, so as to limit its application. Thus, to find a new, no side effects and cytotoxicity of anti-tumor medicine is a main objective for the international medical field.

Osthol, also known as methoxy Parsley phenol, which is named 7-methoxy-8-isopentenyl coumarin, is a Coumarins compound extracted from Umbelliferae plants, and has a known structure of aromatic compounds, having the molecular formula C₁₅H₁₆O₃, molecular weight 244.28, prism-shaped crystal (ether), needle-like crystal (dilute ethanol), mp: 82˜84° C., bp: 145˜150° C., soluble in methanol, ethanol, chloroform, acetone, ethyl acetate and boiling ether, and insoluble in water and petroleum ether. The existing technology shows that this compound has a wide range of pharmacological activities, such as enhancing immune system, anti-osteoporosis, anti-virus, anti-mutative, plant estrogen-like effects, anti-mutagenic, anti-tumor, etc., so that this compound has been highly concern by human.

For the Anti-tumor activity, Kawaiis found that the Osthol shows a obvious antiproliferative effect (Antiproliferative effect of isopentenylate coumarins on serral cancer cell lines, Anticacer Res, 2001, 3 B); Shen Xiu published that the high purified Osthol has great effect in the prevention of tumor, tumor radiation proliferation, and white corpuscle increasing on CN1724529. They choose the cervical cancer in mice (U14), sarcoma (S180), and hepatoma (H22) tumor strains to study the Osthol effect on anti-tumor activity, and through the thymus, spleen and many other indicators of comprehensive survey of the Osthol effect of high anti-tumor activity and low toxicity characteristics. The results shows that the best inhibition tumor rates are U₁₄60.0%, S₁₈₀68.2%, H₂₂62.1%, and Osthol has almost no effect of the liver, spleen index and thymus index. However, the water-solubility of the osthol is poor and limits the applications on the clinical use.

### SUMMARY OF THE PRESENT INVENTION

A main objective of the present invention is to provide a trans-cinnamic acid derivatives hydrolysis from the Osthol and its preparation method and applications thereof.

Accordingly, in order to accomplish the above objective, the present invention provides an anti-tumor compound having a cytotoxic activity, wherein this compound is a new trans-cinnamic acid derivative and its pharmaceutical salts, which is the compound of the formula (I) or its pharmaceutical acceptable salt:

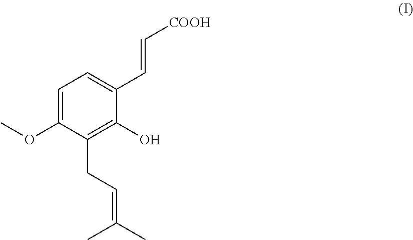

The pharmaceutical acceptable salt is an inorganic salt, such as sodium salt, potassium salt, calcium salt, and magnesium salt, etc.

The pharmaceutical acceptable salt is also an organic salt, such as tromethamine salt, diethanolamine salt, ammonium salt, diethylamine salt, etc.

Another object of the present invention is to provide another three compounds hydrolysis from the Osthol, which is compound I-b, I-c, and I-d respectively.

The structures of the four compounds of the present invention are shown as followings:

I-a: (E)-2-hydroxy-4-methoxy-3-isopentenyl-cinnamic acid

I-b: (Z)-2,4-dimethoxy-3-isopentenyl-cinnamic acid

I-c: (E)-2,4-dimethoxy-3-isopentenyl-cinnamic acid

I-d: 2,4-dimethoxy-3-isopentenyl-cinnamic acid (E:Z=1:1)

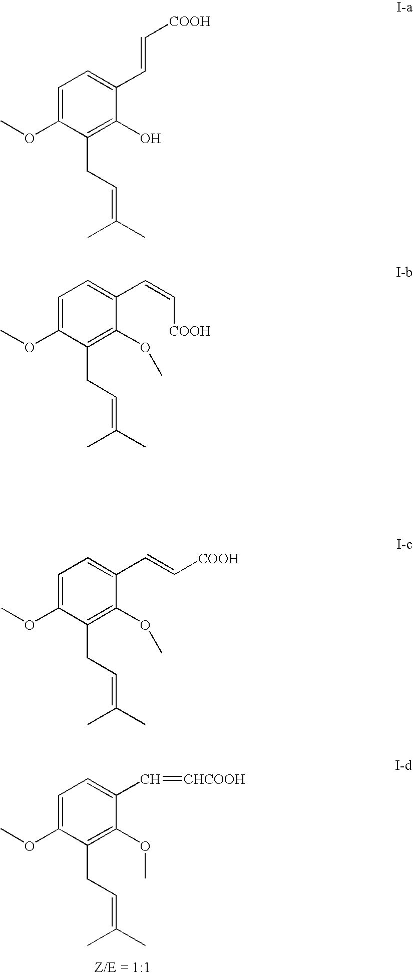

Accordingly, the present invention also provides a preparation method of the compound (I), wherein the Osthol is dissolved into an alkaline solution, heating refluxed, cooling down and then adjusting its pH to 2˜3. Filter the above solution and then use a water content of ethanol for recrystallization to get the compound of type (I).

The alkaline solution is preferably a sodium hydroxide solution.

The concentration of the alkaline solution is preferably from 50% to 70%.

The pH adjusting is using a hydrochloric acid to adjust the pH.

The water content ethanol concentration is preferably from 60% to 80%.

The compound of the formula (I) or its pharmaceutical acceptable salt is able to be applied on the preparation of prevention and/or treatment of tumor medicine.

The tumor is specifically a liver cancer or lung cancer.

The prevention and/or treatment of tumor medicine is any types of medicine, such as injection, freeze-dried powder, orally administered tablets, capsules, pills or granules.

Accordingly, the present invention also provides a preparation method of the compounds I-b, I-c, and I-d respectively.

The compound I-b: dissolve the Osthol into 20% sodium hydroxide solution, stir the solution, heat refluxed the solution for 0.5 hour, cool down the solution to room temperature, drop dimethyl sulfate into the solution, stir the solution for 1 hour at room temperature, then add 20% sodium hydroxide and dimethyl sulfate at the same time, stir the above solution for 0.5 hours, and then heat refluxed for 2 hours, cool down to room temperature, then adjust the pH value to 2˜3 with 1 mol/L hydrochloric acid, filter the above solution, so that a white crystalline-like powder is obtained from 500% ethanol recrystallization.

The compound I-c: dissolve the Osthol into 40% sodium hydroxide solution, stir the solution, drop dimethyl sulfate into the solution at room temperature, stir the solution for 1 hour at room temperature, then add 40% sodium hydroxide and dimethyl sulfate at the same time, stir the above solution for 0.5 hours, and then heat refluxed for 2 hours, cool down to room temperature, then adjust the pH value to 2˜3 with 1 mol/L hydrochloric acid, filter the above solution, so that a white crystalline-like powder is obtained from 50% ethanol recrystallization.

The compound I-d: dissolve the Osthol into 30% sodium hydroxide solution, stir the solution, heat refluxed the solution for 1 hour, cool down the solution to room temperature, drop dimethyl sulfate into the solution, stir the solution for 1 hour at room temperature, then add 30% sodium hydroxide and dimethyl sulfate at the same time, stir the above solution for 1 hours, and then heat refluxed for 1 hours, cool down to room temperature, then adjust the pH value to 2˜3 with 1 mol/L hydrochloric acid, filter the above solution, so that a white crystalline-like powder is obtained from 60% ethanol recrystallization.

The compounds I-b, I-c, and I-d can be applied on medication for tumor prevention and/or treatment medicine.

In vitro test, a method of tetrazolium salt (MTT) is being applied thereon, wherein the MTT method uses a half inhibitory concentration (IC50) as the indicators for a preliminary comparison of the inhibition of four kinds of Osthol hydrolyzate products (I-a, I-b, I-c, I-d) to a variety of tumor cell strains (Hela, BEL-7402, A549, MCF-7/S, U251) and human normal embryonic kidney HEK-293 cells, and screen the compounds which have a good inhibitory effect on tumor cells and low toxicity on normal cells. The experimental results show that: I-a, I-c on Hela and A549 tumor cell have stronger affect for proliferation inhibitory effect; and when the I-a, I-c have 50% inhibitory effect, the normal cells HEK-293 inhibition is unobvious; I-a, I-c anti-tumor activity in vitro is stronger than I-d and I-b, and I-b anti-tumor activity in vitro is the worst within those four compounds.

The vivo experiment used the mice transplanted tumor of liver cancer H22 as a model, and used an inhibition rate as an index, is further applied for the studying of four kinds of Osthol hydrolyzate compounds (I-a, I-b, I-c, I-d) anti-tumor effects. The experimental results show that: whether it is oral or intravenous administration, I-c and I-a anti-tumor effect is clearly superior to I-d and I-b; I-a and I-d intravenous administration can obviously reduce the organ coefficient of tumor-bearing mice thymus, and I-a reducing tumor-bearing mice thymus organ coefficient is significantly less than the group of CTX (cyclophosphamide); I-c and I-d intravenous drug delivery can cause the spleen organ coefficient of the tumor-bearing mice significantly increased; high concentration of I-c and I-d for continuously intravenous drug delivery may cause the mice tail vein and its surrounding tissue be irritating or even corrosive.

A method of mice largest drug delivery is adapted for the process of NIH mice single tail vein administration of acute toxicity test. When the among of each of I-a, I-c, I-b, I-d for NIH mice single tail vein administration is 350 mg/kg, the NIH mice after the drug is delivered are abnormally excited at first, and then become in an inhibit state. Those durations are relatively shorter, the NIH mice had no toxic deaths, and the drug reactions of other groups of animals are similar and no significant differences.

The above results show that the I-a, I-c have better anti-tumor effect, and I-b, I-d have relatively weaker anti-tumor effect; though I-c is slightly better than I-a, I-c compound causes the increased spleen organ coefficient of the tumor-bearing mice, and the high concentration of I-c and I-d for continuously intravenous drug delivery cause the mice tail vein and its surrounding tissue irritating or even corrosive. The I-a and I-c experimental groups both can cause the spleen organ coefficient of the tumor-bearing mice significantly decreased, but significantly less than the positive control group of cyclophosphamide; I-a and I-c have similar results of acute toxicity.

The present invention provides the anti-tumor activity in vivo and in vitro experimental data results of above four compounds I-a, I-b, I-c, I-d having the comparison and conclusion as follows: 1) compound I-a on IC50 of human embryonic kidney cells has higher selectivity of the tumor cells than other compounds; 2) no matter the compounds I-a and I-c in vivo or in vitro, oral or injection have shown a significantly superior to anti-tumor activity of other compounds; 3) compounds I-d and I-c show some stimulation during injection, and the compounds I-a not does not; 4) compounds I-d and I-c can lead to splenomegaly, compounds I-a has no affect. Accordingly, compound I-a has good anti-tumor activity, and meanwhile has relatively higher security for applying on anti-tumor related medicine.

The compounds of the present invention have an important biological activity. The cell cytotoxicity test of five human tumor cells cultured in vivo and in vitro, including human cervical carcinoma Hela cells, human hepatoma BEL-7402 cells, mucinous Human epidermoid A549 lung cancer cells, human breast cancer MCF-7/s cells, human glioma U251 cells and human normal embryonic kidney HEK-293 cell, indicates that the compounds have inhibit effect on tumor cell growth and have almost no effect on normal cells, so as to show the potential to become new antineoplastic medicine.

I-a compound or its pharmaceutical salts and its solvents can be combined with the commonly used pharmaceutical excipients or carriers to have tumor cell growth inhibitory activity, and thus can be used for preparation of tumor prevention and treatment drug combinations. The above drug combinations can be used for injections, tablets, capsules, pills agent, or external liniment; the above drug combinations also can be used for the existing well know method of release or sustained-release formulations pharmaceutical industry, or nano-graded agents.

These and other objectives, features, and advantages of the present invention will become apparent from the following detailed description, the accompanying drawings, and the appended claims.

### DETAILED DESCRIPTION OF THE PREFERRED EMBODIMENT

As described as followings, the compounds I-b, I-c, I-d having similar structure of compound I-a are described as control groups, to further clarify the anti-tumor effects of compound I-a. All the results are shown the form of x̅±SD, and a t-test is applied for comparing statistical differences between groups.

The experimental methods of following embodiments are the conventional methods, if no specific descriptions thereof.

### A First Embodiment

### A Preparation of Compounds

1. A Preparation of I-a Compound:

The scientific name of compound I-a: (E)-2-hydroxy-4-methoxy-3-isopentenyl-cinnamic acid.

Install an electric blender and reflux condenser in a dry, clean 500 ml flask having three openings, add an Osthol 50 g with 300 ml 60% sodium hydroxide aqueous solution, stir the above solution, heat reflux for 8 hours to complete the reaction, then cool down to room temperature, adjust pH value to 2˜3 with 1 mol/L hydrochloric acid, filter the above solution, recrystallize the obtained crystal from the above filtration with 70% ethanol 450 ml, and vacuum dry at 50° C. to obtain a light yellow crystalline powder 36 g, wherein the light yellow crystalline powder yield is 65.7% and mp (melting point) is 92˜93° C.

| | | | |
|---|---|---|---|
| Elemental analysis: theoretical value (%): | C 68.68 | H 6.92 | O 24.40 |
| actual value (%): | C 69.45 | H 6.86 | O 23.69 |

¹H-NMR (400 MHz, CDCl₃) δ 11.1 (1H, s, COOH), 7.89˜8.0 (2H, d, J=8.8 Hz, —CH═CH—COOH), 6.17˜6.85 (2H, d,

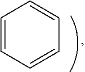

5.1 (1H, s, OH), 3.74 (3H, d, J=7.2 Hz, OCH₃), 1.72 (6H, dt, 2CH₃).

MS: m/z (M⁺+Na) 285, M⁺: 262.10 (100%), M+1: 263.10 (16.5%).

2. A Preparation of I-b Compound:

The scientific name of compound I-b: (Z)-2, 4-dimethoxy-3-isopentenyl-cinnamic acid.

Install an electric blender and reflux condenser in a dry, clean 1000 ml flask having three openings, add the Osthol 60 g with 700 ml 20% sodium hydroxide aqueous solution, stir the above solution, heat reflux for 0.5 hours, then cool down to room temperature, drop dimethyl sulfate 50 ml into the solution, stir the solution for 1 hour at room temperature, then add 20% sodium hydroxide 200 ml and dimethyl sulfate 50 ml at the same time, stir the above solution for 0.5 hours at room temperature, and then heat refluxed for 2 hours, cool down to room temperature, then adjust the pH value to 2˜3 with 1 mol/L hydrochloric acid, filter the above solution, so that a white crystalline-like powder 41 g is obtained from 50% ethanol recrystallization and vacuum dry at 40° C., wherein the white crystalline-like powder yield is 55.4% and mp is 70˜72.5° C.

| | | | |
|---|---|---|---|
| Elemental analysis: theoretical value (%): | C 69.54 | H 7.30 | O 23.16 |
| actual value (%): | C 70.39 | H 6.96 | O 22.65 |

¹H-NMR (400 MHz, CDCl₃) δ 11.1 (1H, s, COOH), 7.88˜8.0 (2H, d, J=8.8 Hz, —CH═CH—COOH), 6.16˜6.82 (2H, d,

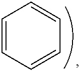

3.73 (6H, d, J=7.2 Hz, 2-OCH₃), 1.72 (6H, dt, 2CH₃).

MS: m/z (M⁺+Na) 299, M⁺: 276 (100%), M+1: 277 (17.7%).

3. A Preparation of I-c Compound:

The scientific name of compound I-c: (E)-2, 4-dimethoxy-3-isopentenyl-cinnamic acid.

Install an electric blender and reflux condenser in a dry, clean 500 ml flask having three openings, add the Osthol 20 g with 250 ml 40% sodium hydroxide aqueous solution, stir and dissolve the above solution, drop dimethyl sulfate 30 ml into the solution, stir the solution for 1 hour at room temperature, then add 40% sodium hydroxide 100 ml and dimethyl sulfate 20 ml at the same time, stir the above solution for 0.5 hours at room temperature, and then heat refluxed for 2 hours, cool down to room temperature, then adjust the pH value to 2˜3 with 1 mol/L hydrochloric acid, filter the above solution, so that a white crystalline-like powder 11 g is obtained from 50% ethanol recrystallization and vacuum dry at 60° C., wherein the white crystalline-like powder yield is 52.2% and mp is 65˜66° C.

| | | | |
|---|---|---|---|
| Elemental analysis: theoretical value (%): | C 69.54 | H 7.30 | O 23.16 |
| actual value (%): | C 68.89 | H 7.52 | O 23.5 |

¹H-NMR (400 MHz, CDCl₃) δ 11.1 (1H, s, COOH), 7.88˜8.0 (2H, d, J=15.5 Hz, —CH═CH—COOH), 6.16˜6.82 (2H, d,

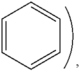

3.73 (6H, d, J=7.2 Hz, 2-OCH₃), 1.72 (6H, dt, 2CH₃).

MS: m/z (M⁺+Na) 299, M⁺: 276 (100%), M+1: 277 (17.7%).

4. A Preparation of I-d Compound:

The scientific name of compound I-d: 2,4-dimethoxy-3-isopentenyl-cinnamic acid (E:Z=1:1).

Install an electric blender and reflux condenser in a dry, clean 500 ml flask having three openings, add the Osthol 30 g with 250 ml 30% sodium hydroxide aqueous solution, stir and dissolve the above solution, heat reflux for 1 hour, cool down to room temperature, drop dimethyl sulfate 40 ml into the solution, stir the solution for 1 hour at room temperature, then add 30% sodium hydroxide 100 ml and dimethyl sulfate 40 ml at the same time, stir the above solution for 1 hours at room temperature, and then heat refluxed for 1 hours, cool down to room temperature, then adjust the pH value to 2˜3 with 1 mol/L hydrochloric acid, filter the above solution, so that a white crystalline-like powder 12.5 g is obtained from 60% ethanol recrystallization and vacuum dry at 60° C., wherein the white crystalline-like powder yield is 31.25% and mp is 96˜97° C.

| | | | |
|---|---|---|---|
| Elemental analysis: theoretical value (%): | C 39.54 | H 7.30 | O 23.16 |
| actual value (%): | C 70.12 | H 7.62 | O 22.26 |

¹H-NMR (400 MHz, CDCl₃) δ 11.1 (1H, s, COOH), 7.88˜8.0 (2H, d, J=11.6 Hz, —CH═CH—COOH), 6.16˜6.82 (2H, d,

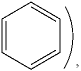

3.73˜3.75 (6H, d, J=7.2 Hz, 2-OCH₃), 1.72 (6H, dt, 2CH₃).

MS: m/z (M⁺+Na) 299, M⁺: 276 (100%), M+1: 277 (17.7%).

The Solubility of compounds I-a, I-b, I-c, and I-d is shown in Table 1.

| | solvent | | | | | |
|---|---|---|---|---|---|---|
| compound | 0.1% NaOH | Methanol | Ethanol | DMSO | Acetone | Chloroform |
| Compound I-a | Soluble | Soluble | Soluble | Soluble | Soluble | Dissolved |
| Compound I-b | Soluble | Soluble | Dissolved | Soluble | Dissolved | Dissolved |
| Compound I-c | Soluble | Soluble | Dissolved | Soluble | Dissolved | Dissolved |
| Compound I-d | Soluble | Soluble | Dissolved | Soluble | Dissolved | Dissolved |

### A Second Embodiment

### An Anti-Tumor Effect In Vitro

1. Experimental Material and Equipments Thereof:

Take 0.1 g of each of the four compounds from the first embodiment, and add 1 ml DMSO to the four compounds respectively for the preparation of 100 mg/ml original solution, and then preserve it at 4° C. A complete culture medium is used for diluting the above original solution into corresponding concentrations for later use.

DMEM culture medium (GIBCO, Invitrogen, USA); fetal bovine serum (FBS; GIBCO, Invitrogen); 100 U/ml penicillin and 100 μg/ml streptomycin (GIBCO, Grand Island, N.Y., USA); methyl thiazolyl blue MTT (thiazolyl blue, Sigma, Mo., USA); trypsin (0.25% Trypsin, GIBCO, Invitrogen); DMSO (100 ml, sigma packaging, Beijing Ding Guo CO.); other reagents are chemical elements.

Human cervical carcinoma Hela cells, human hepatoma BEL-7402 cells, mucinous Human epidermoid A549 lung cancer cells, human breast cancer MCF-7/S cells, human glioma U251 cells, and human normal embryonic kidney HEK-293 cells are purchased from USA, American Type Culture Collection (ATCC). All the tumor cells are used the DMEM medium (containing 10% FBS, 100 U/ml penicillin and 100 μg/ml streptomycin) for culturing and subculturing, and HEK-293 cells are using RPMI1640 medium (10% FBS, 100 U/ml penicillin, and 100 μg/ml streptomycin) for culturing and subculturing, wherein the culture condition is in a 5% CO2 incubator at 37° C.

Microplate reader (USA, Bio-Rad, Model 550); incubator (Thermo Forma, Incubator, USA); centrifuge (HITACHI, RX series, Himac CF 16RX); inverted microscope (leika TE2000, Japan), Thermo adjustable shifting Liquid gun; SW-CJ-IFD single-person single-sided cleaning table (Suzhou Purification Equipment Co., Ltd., NO: 070587); cell culture bottles (Costar, USA), 96-holes cell culture plates (Costar, USA), Delta320 Mettler-Toledo pH device.

2. Anti-Tumor Test In Vitro:

Subculture six kinds of cells, which has a logarithmic growing period, adherent rate about 80%, and in good condition, such as human cervical carcinoma Hela cells, human hepatoma BEL-7402 cells, mucinous Human epidermoid A549 lung cancer cells, human breast cancer MCF-7/S cells, human glioma U251 cells, and human normal embryonic kidney HEK-293 cells. Discard the culture medium, rinse 1-2 times with PBS to remove serum, add 1 ml 0.25% Trypsin-0.01% EDTA (37° C. incubation) to digest for 1-2 minutes, add FBS culture medium to terminate the above digestion, centrifuge at a speed below 800 r/min for 2-3 minutes and discard the supernatant, re-suspended cells in 10% FBS medium, tack the cell suspension liquid for cell counting, adjust the density of the above liquid to 1×10⁵/ml, then inoculate to a 96-holes plates (100 μl/hole), and then incubate in 5% CO2 incubator at 37° C. overnight. The next day add above cells to different concentrations of I-a, I-b, I-c and I-d, 100 μl/hole (its final concentration is respectively 30, 60, 120, 240, 480 μg/ml); meanwhile, set up the blank control group (0 μM), and establish 3 holes. Continuing the cultural process, discard the culture medium after 48 hours that the drug has been effected, add 100 μl PBS of 0.5 mg/ml MTT (PH 7.2) into each holes, after 4 hours culture, remove the culture medium by adherent cells express flipping method, add 100 μl of DMSO to each holes, oscillate it by micro-oscillation device for 5 minutes, and measure the OD value at 490 nm wavelength. Repeat the above experiment three times to get an average value. Calculate the inhibition rate (IR %) of the tumor cell proliferation in vitro from different concentrations of I-a, I-b, I-c, I-d by the following formula:

IR %=(1−ODₛₐₘₚₐₗₑ/OD_{control})×100%

Use SPSS11.5 software to calculate the half inhibitory concentration IC₅₀ of I-a, I-b, I-c and I-d. The results are shown in Table 2 to illustrate IC₅₀ values (unit: μg/ml) of the four compounds.

TABLE 2
Four compounds' IC₅₀ value (unit: μg/ml)

| I-a | | | I-c | | | I-b | | | I-d | | |
|---|---|---|---|---|---|---|---|---|---|---|---|
| 1 | 2 | 3 | 1 | 2 | 3 | 1 | 2 | 3 | 1 | 2 | 3 |

| Hela | 1 | 115 | 96. | 95. | 85.6 | 97. | 331 | 310 | 328 | 144 | 149 | 1 |
|---|---|---|---|---|---|---|---|---|---|---|---|---|
| | 102.54 ± 8.48 | | | 96.23 ± 1.25 | | | 323.6 ± 11.6 | | | 153.59 ± 13.03 | | |
| A549 | 1 | 118 | 107 | 151 | 162. | 159 | 230 | 216 | 205 | 190 | 183 | 1 |
| | 118.39 ± 10.55 | | | 158.06 ± 5.66 | | | 217.68 ± 12.6 | | | 184.56 ± 5.80 | | |
| Bel- | 2 | 237 | 269 | 315 | 300. | 323 | 343 | 357 | 338 | 221 | 211 | 2 |
| | 249.06 ± 17.86 | | | 313.09 ± 11.74 | | | 346.46 ± 10.13 | | | 216.72 ± 5.07 | | |
| U251 | 1 | 173 | 180 | 202 | 192. | 188 | 277 | 284 | 298 | 210 | 193 | 2 |
| | 181.18 ± 7.98 | | | 194.64 ± 7.31 | | | 287.37 ± 9.58 | | | 204.44 ± 9.50 | | |
| MCF-7 | 2 | 216 | 219 | 234 | 229. | 240 | 312 | 317 | 303 | 200 | 196 | 1 |
| | 224.90 ± 11.86 | | | 234.67 ± 5.68 | | | 311.07 ± 7.31 | | | 193.46 ± 7.57 | | |
| HEK- | 3 | 399 | 414 | 383 | 367. | 356 | 358 | 371 | 366 | 347 | 366 | 3 |

| | | | |
|---|---|---|---|
| 404.07 ± 9.20 | 369.49 ± 13.58 | 365.49 ± 6.14 | 350.77 ± 14.09 |

The I-a, I-c, I-d compounds have greater effect on the tumor cell proliferation inhibition of the five strains than the I-b, and greater effect on Hela and A549 cells, and less effect of inhibition on the HEK-293 cells. The HeLa cell proliferation IC₅₀ values of I-a, I-c, I-b and I-d at 48 hours are 102.54±8.48, 96.23±1.25, 323.6±11.6 and 148.59±5.96 μg·mL⁻¹ respectively; the A549 cell proliferation IC₅₀ values of I-a, I-c, I-b and I-d at 48 hours are 118.39±10.55, 158.06±5.66, 217.68±12.6 and 184.56±5.80 μg·mL⁻¹ respectively. In conclusion, the anti-tumor activities in vitro of I-a and I-c are greater than I-d and I-b, wherein I-b has the worst anti-tumor activity in vitro. The four compounds of the present invention have greater IC₅₀ values on the HEK-293 cells. The HEK-293 cells IC₅₀ values of I-a and I-c are 404.07±9.20 and 369.49±13.58 μg·mL⁻¹ respectively, which have significantly greater IC₅₀ value than the IC₅₀ value of tumor cells, and when the concentrations of I-a, I-c have 50% inhibitory effect on the tumor cells, the inhibitory effect on HEK-293 cells is unobvious. To sum up, I-a, I-c in vitro anti-tumor activities in vitro are relatively stronger, have selectivity on Hela and A549 cells, and have weaker effect on the cell proliferation activities of human normal embryo kidney HEK-293 cells, wherein the HEK-239 50% inhibitory effect concentration IC₅₀ of I-a is 404.07 μg·mL⁻¹, which is much larger than the IC₅₀ of the tumor cells, so as to show the higher selectivity on tumor cells.

### A Third Embodiment

### Anti-Tumor Effect In Vivo

Most of the drugs currently used for cancer chemotherapy are found from the animal transplanted tumor test, and compare this method to vitro cell screening method, the advantages of animal transplanted tumor method is after the inoculation a certain amount of tumor cells or the cell-less filtrate (virus tumor) can make a whole group of animals carry the same tumor cells, have the same growing rate, and have smaller individual differences; the survival rate after inoculation is 100%; the affects on the hosts are similar, and easer to objectively judge the efficacy of the therapy; can be continuously transplanted in the same species or genus to long-term reserve the tumor cells for test purposes; the test periods are relatively shorter. Therefore, the transplanted tumor is currently adapted for most of the anti-tumor drugs screening test.

The third embodiment of the present invention based on a screening in vitro adapts hepatoma cell strain H₂₂ for making a mice tumor model, and further confirm the anti-tumor effect of the compound I-a.

1. Experimental Material and Equipments Thereof:

Take 0.1 g of each of the four compounds from the first embodiment, dissolve 0.1 g of each of four compounds into 0.1 mol/L NaOH (saline preparation), adjust the pH value to 7.5 by 0.1 mol/L HCl, add saline into each of the above solution for adjusting to appropriate concentration, and filter the above solutions by a microporous filter (pore size: 0.22 um) for tail vein injection; take 0.1 g of each of the four compounds from the first embodiment, respectively dissolve the four compounds by saline to a concentration of the administration of the drugs for oral medication; prepare an injection use cyclophosphamide (Jiangsu Henry Products Pharmaceutical Company Limited, referred as: CTX) with saline to normal administration concentration, prepare when the CTX is needed, and avoid light.

The mice ascites hepatoma cell strain H22 is purchased from the Experimental Center of Zhongshan University.

SPF level Kunming mice (half of the mice are male and another half are female), having the weight around 18˜22 g, are purchased from the Guangdong Provincial Medicine Laboratory Animal Experimental Center.

Microplate reader (United States Bio-Rad, Model 550); incubator (Thermo Forma, Incubator, USA); centrifuge (HITACHI, RX series, Himac CF 16RX); inverted microscope (leika TE2000, Japan), Thermo adjustable shifting Liquid gun; SW-CJ-IFD single-person single-sided cleaning table (purification equipment Co., Ltd Xuzhou, NO: 070587); cell culture bottles (Costar, USA), 96-holes cell culture plates (Costar, USA), Delta320 Mettler-Toledo pH device.

2. Anti-Tumor Test In Vivo:

(1) Preparation of Mice Tumor Model

(1.1) H22 Subculture In Vivo:

Recovery the mice ascites hepatoma cell strain H22, put the cell suspension liquid into a centrifugal tube and use the 4° C. saline to wash the suspension liquid twice, then centrifuge to discard the supernatant, add appropriate 4° C. saline to dilute the suspension liquid, count cells with 0.2% trypan blue, adjust the density to 10⁷ cell/ml, according to 0.2 (ml/mice) intraperitoneal inoculation H22 cell suspension liquid.

(1.2) Armpit Inoculation:

After ten days of the intraperitoneal inoculation, the cervical vertebra is dislocated. Disinfect the mice abdominal skin, and suck the milky ascites with a sterile syringe, then adjust the concentration of tumor cells to 1×10⁷ cell/ml with injection used saline. Disinfect the right armpit skin of the Kunming mice with alcohol swab, subcutaneously vaccinate the cell suspension liquid 0.2 ml, conventional feeding. The results show that after the mice inoculated the ascitic hepatoma H22 cell strain, the subcutaneous tumor can be touched after 3 to 4 days of the inoculation. Totally there are 180 tumor-bearing mice.

(2) Drug Delivery of the Mice Model:

The 180 tumor-bearing mice are divided into 18 groups by the weigh of the mice, wherein each group has 10 mice (half male and half female). The drug delivery methods of each group are shown in Table 3 to illustrate eighteen groups of mice drug delivery situation.

| | | | |
|---|---|---|---|
| 1 | Model group | 2 | Cyclophosphamide group: |
| | | | drug delivery 60 mg/kg |
| 3 | I-a tail intravenous: 25 mg/kg | 4 | I-a tail intravenous: 50 mg/kg |
| 5 | I-a tail intravenous: 100 mg/kg | 6 | I-a oral enteral: 200 mg/kg |
| 7 | I-b tail intravenous: 25 mg/kg | 8 | I-b tail intravenous: 50 mg/kg |
| 9 | I-b tail intravenous: 100 mg/kg | 10 | I-b oral enteral 200 mg/kg |
| 11 | I-c tail intravenous: 25 mg/kg | 12 | I-c tail intravenous: 50 mg/kg |
| 13 | I-c tail intravenous: 100 mg/kg | 14 | I-c oral enteral 200 mg/kg |
| 15 | I-d tail intravenous: 25 mg/kg | 16 | I-d tail intravenous: 50 mg/kg |
| 17 | I-d tail intravenous: 100 mg/kg | 18 | I-d oral enteral 200 mg/kg |

Group 1 is the model group, and the mice of group 1 are given the tail vein saline injection daily started on the second day of the tumor-bearing for 10 days.

Group 2 is the cyclophosphamide (CTX) group, and the mice of group 2 are only given the cyclophosphamide in the intraperitoneal of the mice on the second day of the tumor-bearing.

Group 3 to 18 groups are given the four compounds respectively, wherein the groups delivered 25 mg/kg drug are tail vein low-dose group, wherein the groups delivered 50 mg/kg drugs are the tail vein medium-dose group, wherein the groups delivered 100 mg/kg drugs are the tail vein high-dose group, wherein the groups delivered 200 mg/kg drugs are the oral enteral group. These mice from group 3 to 18 start the drug delivery on the second day of the tumor-bearing, and delivered once a day for 10 sequent days.

The above drug deliveries are 20 ml/kg.

(3) General Observations:

The observation by neck eyes of the tumor volumes of the mice from the four compounds groups are significantly smaller than the tumor volumes of the mice from the model group after 6 to 8 days. When the tumor is peeling off, the tumor from the model group is significantly increased in size, the boundaries are unclear, the texture is soft, the tumor is hard to peel off, and some tumors are spray out to the sternum and clavicle; the groups that delivered drugs has smaller spraying area, the depth is smaller, and the tumor is easier to peel off.

During the drug delivery process, the mice of the I-a high-dose group are excited after the injection; after 2-3 minutes, the activity of the mice starts decreasing; after 10 minutes, the mice are recovered again, and then continuously delivering drugs for 10 days, the mice does not have any death; This phenomenon of central nervous excitement at first then being inhibited can also be observed in the I-c high-dose group. Ib and Id high-dose groups have the central nerve inhibition phenomenon, and imply that I-b and I-d may have a certain inhibition effect. The oral dose group has no abnormalities of animal general observation. I-c and I-d high dose group are observed blockage point in the mice tail after 3 days, and the mice start appearing black tail and necrosis after 6 days, and then the end point of the mice tail is broken since the necrosis, wherein the result implies that the I-c and I-d have irritation or corrosive near the tail vein of the mice and its surrounding tissue.

(4) Tumor Inhibition Effect:

After finishing the drug delivery process, weight the mice, then kill the mice by pulling off the neck, and anatomy to get the tumor tissue of the mice on the next day of finishing the drug delivery process, and then weight the anatomized tumor by electronic weighing scales to calculate the inhibition rate.

$$Inhibition Rate ( \% ) = ( 1 - \frac{\begin{matrix}tumor average weight \\ of drug delivery group\end{matrix}}{\begin{matrix}tumor average weight \\ of control group\end{matrix}} ) \times 100$$

The results are shown in Table 4 to illustrate the compounds hydrolyzed from Osthol and its inhibition rate (IR) of mice H₂₂ liver cancer.

TABLE 4
Compounds hydrolyzed from Osthol and its inhibition rate (IR) of mice
H₂₂ liver cancer

| | | | | Mice weight/g | | | | | |
|---|---|---|---|---|---|---|---|---|---|
| | | | Drug | After | Before | | | | |
| | Drug | dose/mg · | delivery | drug | drug | | | Thymus index/ | Spleen index/ |
| Group | delivery | (kg · d)⁻¹ | time | delivery | deli. | Tumor weight/g | IR/% | mg · g⁻¹ | mg · g⁻¹ |
| Model | i.v. | — | 10 | 19.9 ± 2.0 | 23.8 ± 2.3 | 1.533 ± 0.443 | — | 3.66 ± 0.74 | 3.75 ± 0.69 |
| CTX | ip. | 60/single | 10 | 19.6 ± 2.0 | 23.6 ± 1.8 | 0.686 ± 0.303** | 55.2 | 2.46 ± 0.65** | 3.00 ± 0.59* |
| I-a | i.v. | 25 | 10 | 20.8 ± 2.6 | 24.8 ± 2.6 | 1.398 ± 0.355 | 8.8 | 3.21 ± 0.68# | 3.49 ± 0.57 |
| | i.v. | 50 | 10 | 20.2 ± 2.1 | 25.4 ± 2.9 | 1.047 ± 0.342* | 31.7 | 3.02 ± 0.45*# | 4.12 ± 0.95## |
| | i.v. | 100 | 10 | 21.1 ± 2.2 | 25.1 ± 1.8 | 0.775 ± 0.381** | 49.4 | 2.95 ± 0.28*# | 3.60 ± 0.53# |
| | i.g. | 200 | 10 | 19.9 ± 2.1 | 25.6 ± 2.6 | 0.907 ± 0.424** | 40.8 | 3.30 ± 0.99# | 4.32 ± 1.35# |
| I-c | i.v. | 25 | 10 | 20.3 ± 2.0 | 24.3 ± 2.2 | 1.322 ± 0.278 | 13.7 | 3.24 ± 0.53# | 3.57 ± 0.72 |
| | i.v. | 50 | 10 | 20.0 ± 1.9 | 25.8 ± 2.4 | 0.875 ± 0.379** | 42.9 | 3.15 ± 0.86 | 3.63 ± 0.76 |
| | i.v. | 100 | 10 | 20.1 ± 1.9 | 25.7 ± 2.1 | 0.782 ± 0.368** | 49.0 | 3.10 ± 0.66# | 5.34 ± 1.40**## |
| | i.g. | 200 | 10 | 20.1 ± 1.8 | 25.2 ± 3.0 | 0.866 ± 0.489** | 43.5 | 3.40 ± 1.05# | 4.12 ± 1.21# |
| I-b | i.v. | 25 | 9 | 20.4 ± 2.5 | 24.8 ± 2.6 | 1.367 ± 0.524 | 10.8 | 3.24 ± 0.53# | 3.93 ± 0.55## |
| | i.v. | 50 | 10 | 20.3 ± 2.3 | 24.4 ± 2.0 | 1.152 ± 0.47 | 24.8 | 3.15 ± 0.86 | 3.82 ± 0.78# |
| | i.v. | 100 | 10 | 20.7 ± 2.6 | 24.7 ± 3.0 | 0.911 ± 0.417* | 40.6 | 3.10 ± 0.66# | 4.01 ± 1.01# |
| | i.g. | 200 | 10 | 20.1 ± 2.2 | 25.8 ± 2.0 | 1.108 ± 0.364* | 27.7 | 3.40 ± 1.05# | 3.99 ± 0.57## |
| I-d | i.v. | 25 | 10 | 20.6 ± 2.7 | 23.9 ± 2.3 | 1.307 ± 0.436 | 14.7 | 3.23 ± 0.59# | 3.95 ± 1.16# |
| | i.v. | 50 | 10 | 19.9 ± 1.7 | 24.7 ± 3.0 | 1.068 ± 0.437* | 30.3 | 3.05 ± 0.60# | 3.63 ± 0.76 |
| | i.v. | 100 | 10 | 19.8 ± 2.0 | 24.7 ± 3.0 | 0.901 ± 0.425** | 41.2 | 2.90 ± 0.35** | 5.27 ± 1.50**## |
| | i.g. | 200 | 10 | 20.0 ± 2.1 | 24.6 ± 2.9 | 0.991 ± 0.410* | 35.3 | 3.21 ± 0.93# | 4.06 ± 0.82## |

*represents comparing to model group:
*p < 0.05,
**p < 0.01;
#with CTX groups
#p < 0.05,
##p < 0.01

(4.1) I-a Inhibition Effect on Mice Tumor:

After finishing the drug delivery process, the tumor-bearings of each group grow over than 1 g. The mice tumor weight of I-a drug delivery group is significantly reduced. Compare the medium, high-dose, and oral enteral drug delivery group to the model group, there are obvious differences, wherein the inhibition rate of high-dose group is about 50%, and the inhibition rate of the oral enteral group is over 40%.

(4.2) I-c Inhibition Effect on Mice Tumor:

After finishing the drug delivery process, the tumor-bearings of each group grow over than 1 g. The mice tumor growth of I-c drug delivery group is significantly inhibited. The tumor weight of the drug delivery group is significantly lower than the model group. Compare the tumor weight of medium, high-dose, and oral enteral drug delivery group to the model group, there are obvious differences (**p<0.01), wherein the inhibition rate of medium-dose group is larger than 40%, the inhibition rate of high-dose group is about 50%, and the inhibition rate of the oral enteral group is over 40%.

(4.3) I-b Inhibition Effect on Mice Tumor:

An observation is found during the experimental process that the mice tumor of I-b drug delivery group is not significantly inhibited, and compare the I-b drug delivery group to the model group, only high-dose and oral enteral group are significantly inhibited (*p<0.05), wherein the inhibition rate of high-dose group is higher than 40%, and the inhibition rate of the oral enteral group is lower than 30%.

(4.4) I-d Inhibition Effect on Mice Tumor:

After finishing the drug delivery process, the tumor-bearings of each group grow over than 1 g. The mice tumor growth of I-d drug delivery group is significantly inhibited. The tumor weight of the drug delivery group is significantly lower than the model group. Compare the high-dose and oral enteral drug delivery group to the model group, there are obvious differences (**p<0.01), wherein the inhibition rate of high-dose group is larger than 40%, and the inhibition rate of the oral enteral group is over 30%.

From the aspect of mice weight increasing from the beginning of inoculation to the end of the test, the mice weight of the drug delivery group of the four compounds increase more than model and CTX group, wherein the weight increasing of I-a and I-c vein drug delivery, high-dose, and oral enteral group is dominated. No matter vein drug delivery or oral drug delivery, the strength of the four compounds anti-tumor effect on the tumor model, in accordance of the order of strong to weak, is as following: I-c>I-a>I-d>I-b. No matter vein drug delivery or oral drug delivery, the inhibition rate of I-c and I-a are over 40%, which achieves the effective judgment of anti-tumor study (the requirement is comparing to the model group, the inhibition rate has to be at least 40%; meanwhile, the study has to be statistical significance, which is P<0.05).

(5) Spleen Index and Thymus Index Calculation:

Thymus and spleen are respectively the main central immune organs and peripheral immune organs, which can certainly express the status of immune system, and the spleen index and thymus index directly reflect the body immune level. The high spleen index causes the Splenomegaly and side effects, and the low thymus index shows the inhibition on the thymus and side effects.

After the drug delivery, weight the mice weight, then execute the mice, and then weight the spleen and thymus weight by electronic weighting scale respectively. The spleen index and thymus index are the weight (mg) of mice spleen, thymus of each group/mice weight (g).

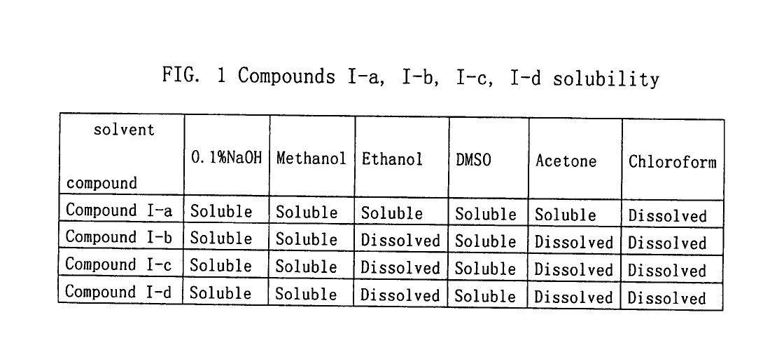

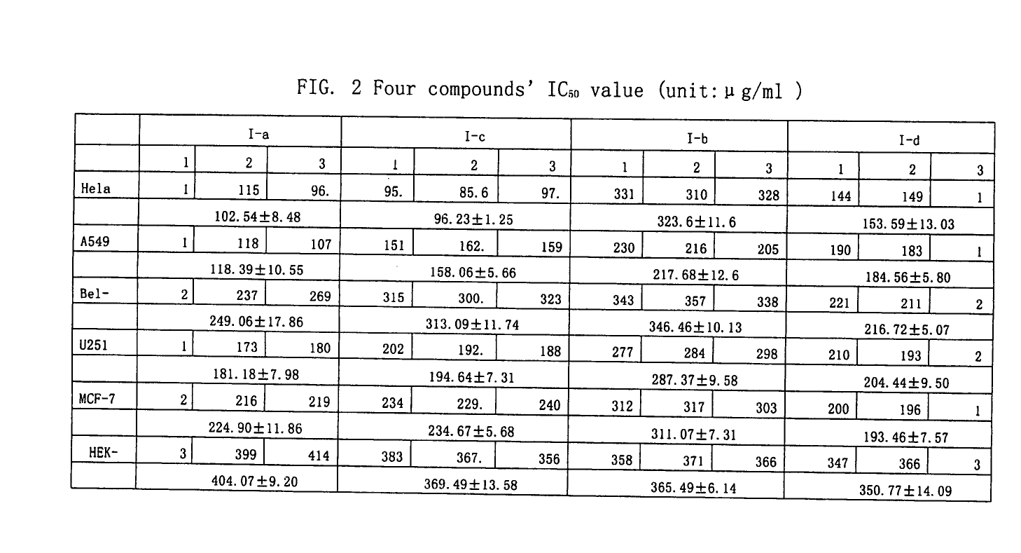

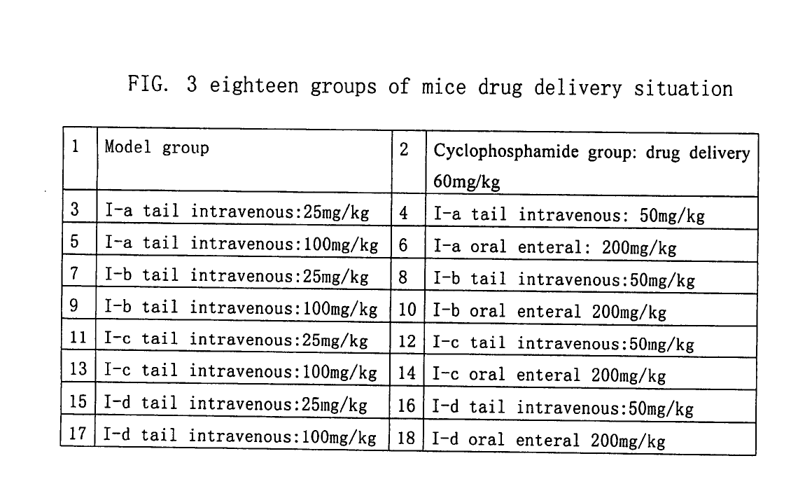

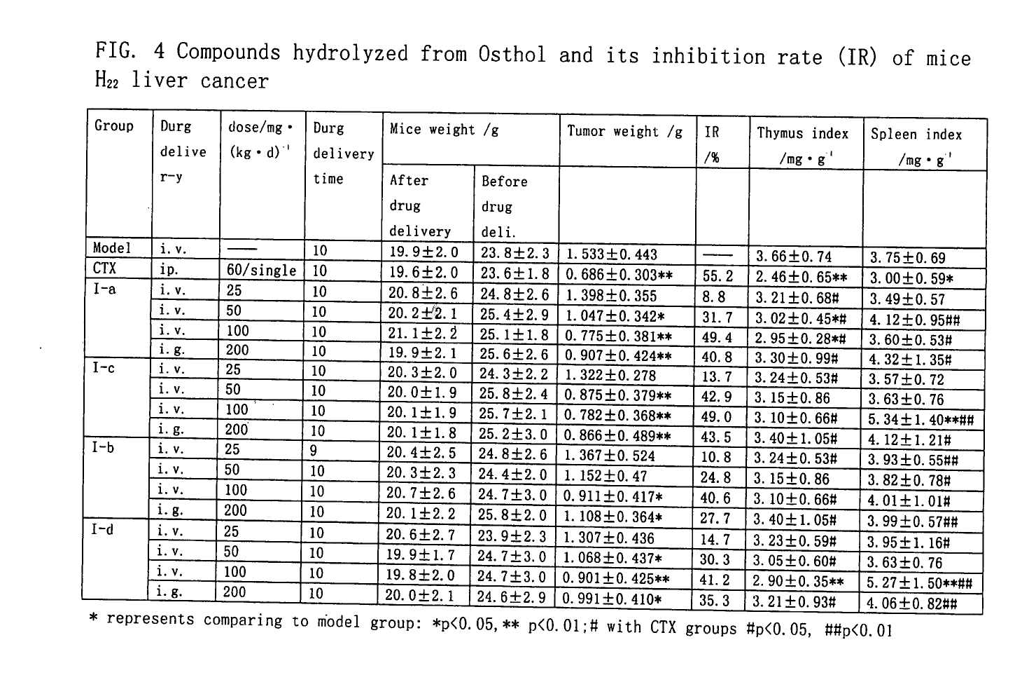

The results are shown in FIG. 4.

(5.1) The Mice Spleen Index and Thymus Index of I-a Group:

Mice spleen index of medium and high-dose group, comparing to the model group, is decreased (*p<0.05), but comparing to CTX group, the thymus index of each of the dose group are significantly higher, and has significant differences (#p<0.05); the spleen index of each of the dose group has no significant differences comparing to the tumor-bearing control group, and are significantly higher than CTX group.

(5.2) The Mice Spleen Index and Thymus Index of I-c Group:

The thymus index and spleen index of each dose group are significantly higher than CTX group, but compare to the model group, there is no significant differences.

(5.3) The Mice Spleen Index and Thymus Index of I-b Group:

The thymus index and spleen index of each dose group are no significant differences compared to the model group.

(5.4) The Mice Spleen Index and Thymus Index of I-d Group:

The thymus index of high-dose group mice, compared to model group mice, is significantly dropped (**p<0.01), and the spleen index of high-dose group mice, compared to model group mice, is significantly increased (**p<0.01).

From the aspect of the influence of thymus organ index, CTX can significantly decrease the thymus index (p<0.01). In the I-a vein drug delivery test, the high-dose group can significantly decrease the thymus index (p<0.05), but the influence of the high-dose group on decreasing the thymus index is significantly lighter than cyclophosphamide group (compared to CTX group, p<0.05). The high-dose group of I-d vein drug delivery can significantly decrease the thymus organ index (p<0.01). The thymus index of oral enteral and other drug delivery groups, compared to the model group, have no significant differences (p<0.05). From the aspect of the influence of spleen organ index, the high-dose groups of I-c and I-d vein drug delivery significantly increase the spleen index (p<0.01), and the spleen index of oral enteral and other drug delivery groups, compared to model groups, have no significant differences (p>0.05). Those results show that I-a and I-c vein drug delivery can certainly inhibit the tumor-bearing mice thymic immunity, but the inhibition ability of the tumor-bearing mice thymic immunity of I-a and I-c vein drug delivery is lower than CTX group. I-c and I-d vein drug delivery can cause the splenomegaly of the tumor-bearing mice.

(6) Other Experimental Results:

From the aspect of mice weight increasing from the beginning of inoculation to the end of the test, the mice weight of the drug delivery group of the four compounds increase more than model and CTX group (60 mg/kg, single drug delivery on the second day after the inoculation), wherein the weight of mice from the vein drug delivery of I-a and I-c medium and high-dose groups has increased most. In the mice tail vein drug delivery process, the I-c and I-d high-dose group are observed blockage point in the mice tail after 3 days, and the mice start appearing black tail and necrosis after 6 days, and then the end point of the mice tail is broken since the necrosis, wherein the result shows that the high-dose I-c and I-d have irritation or corrosive near the tail vein of the mice and the surrounding tissue thereof.

### A Fourth Embodiment

### Acute Toxicity Test

1. Experimental Material:

Take 0.1 g of each of the four compounds from the first embodiment, dissolve 0.1 g of each of four compounds into 0.1 mol/L NaOH (saline preparation), adjust the pH value to 8 by 0.1 mol/L HCl, add saline into each of the above solution for adjusting to appropriate concentration, and filter the above solutions by a microporous filter (pore size: 0.45 um) for tail vein injection; prepare the above solution when is about to use, and avoid light.

SPF level NIH mice (half male and half female), having the weight around 18˜22 g, are purchased from the Guangdong Provincial Medicine Laboratory Animal Experimental Center.

2. Acute Toxicity Test:

Select 100 NIH qualified mice, and randomly and equally separate the mice into five groups, which are saline control group, I-a, I-c, I-b, and I-d group. The mice from each group are given once corresponding drug subjects respectively, wherein each drug delivery volume is 20 ml/kg, and the concentration of the four compounds is 17.5 mg/ml, which means each compound dosage is 350 mg/kg.

After the drug delivery, continuously observe the mice responses for 4 hours; after all, observe the mice twice a day (one is in the morning, and the other is in the afternoon) for 14 days, wherein the observation includes the appearance, behavior, secretions, excreta of the mice etc., and recording all the death situation, poisoning symptoms, the starting time and ending time of toxic reactions of the mice, severity, duration time of toxic, and to see if it is reversible reaction etc., sent it to the pathology examination if necessary, weight the mice before and after the drug delivered day of 3, 7, 10, and 14 days. After finishing the observation process, execute all of the mice, and anatomize the dead bodies of mice.

Accordingly, the mice of saline control group are all healthily alive after the experiment, gained weight, and no abnormal responses. The mice of the four compounds (I-a, I-c, I-b, I-d) drug delivery has the following responses: after the drug delivery, the mice immediately have the situations of jumping, running in the cage, and extremely excited, and then running unstably. After 2 to 4 minutes, the mice activities are decreased, creeping, walking slowly when stimulating it, and walking unstably. After 10 to 15 minutes, the activities and movement of the mice are gradually returned to normal, the mice responses are similar between each group and have no significant differences, and no other abnormal behaviors is observed. From the second to 14^{th} days, all the testing mice are normally being supplied food and water, and the mice responses are normal, and no any abnormal behaviors are found. All the mice are survived, and no abnormal observation by necked eyes after the anatomy of the dead bodies.

The result shows that in the single tail vein drug delivery, the maximum duration of the drug among of the NIH mice is larger than 350 mg/kg.

### INDUSTRIAL APPLICATIONS

The compound I-a is a new cinnamic acid derivative having a better anti-tumor activity and lower toxic. The compound I-a has a great potential to become a new anti-tumor medication. The preparation method of the cinnamic acid derivatives, having the characteristic of selectively inhibiting tumor cells and minimum toxic to organic creatures, gives a firmly fundamental, and has important potential of developing the industrialization, and great contributions to the human researches of anti-tumor medication. The preparation method of the present invention provides a safe raw materials, simple equipments, and simple producing method, such that the present invention has high potential market prospects.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

1-10. (canceled)

11. A pharmaceutically acceptable salt, comprising: a chemical compound chemically reacting with alkaline solution, wherein said chemical compound has a chemical structure of:

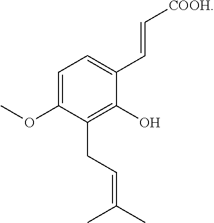

12. The pharmaceutically acceptable salt, as recited in claim 11, which is an inorganic salt selected from a group consisting of sodium salt, potassium salt, calcium salt, and magnesium salt.

13. The pharmaceutically acceptable salt, as recited in claim 11, which is an organic salt selected from a group consisting of tromethamine salt, diethanolamine salt, ammonium salt, and diethylamine salt.

14. The pharmaceutically acceptable salt, as recited in claim 11, wherein said alkaline solution is a sodium hydroxide solution.

15. The pharmaceutically acceptable salt, as recited in claim 12, wherein said alkaline solution is a sodium hydroxide solution.

16. The pharmaceutically acceptable salt, as recited in claim 13, wherein said alkaline solution is a sodium hydroxide solution.

17. The pharmaceutically acceptable salt, as recited in claim 14, wherein a concentration of said sodium hydroxide solution is 50%˜70%.

18. The pharmaceutically acceptable salt, as recited in claim 15, wherein a concentration of said sodium hydroxide solution is 50%˜70%.

19. The pharmaceutically acceptable salt, as recited in claim 16, wherein a concentration of said sodium hydroxide solution is 50%˜70%.

20. A method for preparing said chemical compound of said structure (I), comprising the steps of:
(a) dissolving Osthol into an alkaline solution,
(b) heating above said solution for refluxing,
(c) cooling down above said solution to adjust a pH value to 2˜3,
(d) filtering above said solution, and
(e) re-crystallizing with ethanol contenting water to obtain said chemical compound of said structure (I), wherein said chemical compound has a chemical structure of:

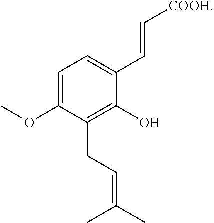

21. The method, as recited in claim 20, wherein said alkaline solution is a sodium hydroxide solution, wherein HCL solution is applied thereto for adjusting pH value of said step of cooling down.

22. The method, as recited in claim 21, wherein a concentration of said sodium hydroxide solution is 50%˜70%.

23. The method, as recited in claim 20, wherein a concentration of said ethanol contenting water is 60% to 80%.

24. The method, as recited in claim 21, wherein a concentration of said ethanol contenting water is 60% to 80%.

25. The method, as recited in claim 22, wherein a concentration of said ethanol contenting water is 60% to 80%.

26. The chemical compound, having a chemical structure of:

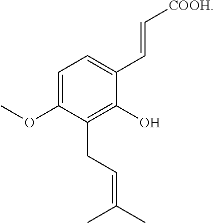

27. The chemical compound, as recited in claim 26, wherein said chemical compound is applied on a tumor prevention and/or therapy medication.

28. The chemical compound, as recited in claim 27, wherein said tumor is selected from the group consisting of liver cancer and lung cancer.

29. The chemical compound, as recited in claim 27, wherein said tumor prevention and/or therapy medication is provided in an administered form selected from the group consisting of injection, a lyophilized powder, an orally administered tablet, a capsule, and a granule.

30. The chemical compound, as recited in claim 28, wherein said tumor prevention and/or therapy medication is provided in an administered form selected from the group consisting of injection, a lyophilized powder, an orally administered tablet, a capsule, and a granule.
